(19) United States
(12) Patent Application Publication
(10) Pub. No.: US 2026/0340821 A1
(43) Pub. Date: Oct. 29, 2026

(54) Method and Apparatus for Simulating Blood Perfusion, and Electronic Device

(71) Applicant: Siemens Healthineers AG, Forchheim (DE)

(72) Inventor: Xi Zhao, Shanghai (CN)

(73) Assignee: Siemens Healthineers AG, Forchheim (DE)

(21) Appl. No.: 19/657,724

(22) Filed: Apr. 24, 2026

(30) Foreign Application Priority Data
Apr. 27, 2025 (CN) 202510546669.9

(51) Int. Cl. G06T 7/00; A61B 6/00; A61B 6/03; A61B 6/50; G06T 5/70

(52) U.S. Cl. CPC G06T 7/0012; A61B 6/03; A61B 6/504; A61B 6/5217; G06T 5/70; G06T 2207/10081; G06T 2207/20081; G06T 2207/30016; G06T 2207/30104

(57) ABSTRACT

Techniques are provided for simulating blood perfusion. The techniques include acquiring multiphase CT vascular imaging data of target tissue and extracting multiple time sequence vascular images. Based on the multiple time sequence vascular images, vascular structure information of the target tissue is extracted and a time sequence CT value of the target tissue is determined. A corresponding blood perfusion image and a perfusion parameter are generated by inputting the vascular structure information and the time sequence CT value into a pre-trained blood perfusion prediction model. The present disclosure helps to reduce a patient's radiation exposure, reduce the cost of medical treatment, and improve the imaging efficiency of blood perfusion images.

### CROSS-REFERENCE TO RELATED APPLICATIONS

This application claims priority to China patent application no. CN 202510546669.9, filed on Apr. 27, 2025, the contents of which are incorporated herein by reference in their entirety.

### TECHNICAL FIELD

The present disclosure relates to the technical field of CT imaging and, in particular, to a method for simulating blood perfusion, an apparatus for simulating blood perfusion, and an electronic device.

### BACKGROUND

Computed Tomography (CT) is a medical imaging technology that uses an X-ray beam to scan the human body in layers and can generate detailed images of the internal structure of the body with the aid of computer processing. The principles of CT mainly comprise, based on the absorption and attenuation characteristics of X-rays penetrating human tissue, receiving attenuated signals at different angles which are recorded by a detector after a transmitted X-ray beam has penetrated the human body. Since different tissues absorb X-rays to different extents, the attenuated signals received by the detector will vary. Based on these attenuated signals, a CT value of each voxel in a cross section of the interior of the body can be calculated and the CT value can be used to further reconstruct a cross-sectional image of the interior of the body.

Through the use of computed tomography, CT angiography (CTA) can be performed to examine abnormalities in blood vessel morphology or vascular structure of specific tissues of a patient, e.g. vascular stenosis, aneurysms, vascular malformation, etc.

In addition, CT can be used to perform CT perfusion (CTP) imaging so as to obtain information relating to blood perfusion in specific tissues of a patient, to assess blood flow function in the patient, and to assist in precise diagnosis and treatment of vascular disease.

However, CT perfusion imaging involves a high radiation dose, expensive imaging equipment, and has low imaging efficiency. Therefore, CT perfusion imaging examination is unsuitable for the vast majority of patients in clinical settings.

### SUMMARY

Given the aforementioned drawbacks of conventional CT systems, a method of simulating blood perfusion is needed to reduce a patient's radiation exposure, reduce the cost of medical treatment, and to improve the imaging efficiency of blood perfusion images.

In view of the above, according to one aspect of the present disclosure, a method for simulating blood perfusion is provided, comprising: acquiring multiphase CT vascular imaging data of target tissue, and extracting multiple time sequence vascular images; based on the multiple time sequence vascular images, extracting vascular structure information of the target tissue, and determining a time sequence CT value of the target tissue; and inputting the vascular structure information and the time sequence CT value into a pre-trained blood perfusion prediction model, so as to generate a corresponding blood perfusion image and a perfusion parameter.

According to another aspect of the present disclosure, an apparatus for simulating blood perfusion is provided, the apparatus comprising: a first unit, configured to acquire multiphase CT vascular imaging data of target tissue, and extract multiple time sequence vascular images; a second unit, configured to, based on the multiple time sequence vascular images, extract vascular structure information of the target tissue, and determine a time sequence CT value of the target tissue; and a third unit, configured to input the vascular structure information and the time sequence CT value into a pre-trained blood perfusion prediction model, so as to generate a corresponding blood perfusion image and a perfusion parameter.

According to another aspect of the present disclosure, an electronic device is provided, comprising: at least one processor; and at least one memory in communicative connection with the at least one processor; wherein the at least one memory stores an instruction executable by the at least one processor, the instruction being executed by the at least one processor to enable the at least one processor to perform a method for simulating blood perfusion.

It should be understood that the content described in this section is not intended to limit the scope of the present disclosure. Additional and alternate features of the present disclosure will become apparent through the following specification.

### BRIEF DESCRIPTION OF THE DRAWINGS

The drawings, which illustratively show embodiments and form part of this specification, are used together with the textual description of this specification to explain exemplary ways of implementing embodiments. The embodiments shown are provided for exemplary purposes and are not intended to limit the scope of the claims. In all of the drawings, identical reference signs denote similar but not necessarily identical key elements.

Embodiments of the present disclosure will be described in detail below with reference to the drawings, to give those skilled in the art a clearer understanding of the abovementioned and other features and advantages of the present disclosure. In the drawings:

FIG. 1 illustrates a flowchart of an example method for simulating blood perfusion according to embodiments of the present disclosure;

FIG. 2 illustrates a flowchart of an example method for training a blood perfusion prediction model according to embodiments of the present disclosure;

FIG. 3 illustrates a block diagram of the structure of an example apparatus for simulating blood perfusion according to embodiments of the present disclosure; and

FIG. 4 illustrates a block diagram of an example electronic device according to embodiments of the present disclosure.

### DETAILED DESCRIPTION

Illustrative embodiments of the present disclosure will be described below with reference to the drawings, including various details of embodiments of the present disclosure to aid understanding, and these should be regarded as merely illustrative. Therefore, it should be appreciated by those skilled in the art that various changes and modifications could be made to the embodiments described here without deviating from the scope of the present disclosure. Likewise, for clarity and conciseness, descriptions of commonly known functions and structures are omitted in the following descriptions.

As stated above, a specialist can use computed tomography to perform CT perfusion imaging so as to obtain information relating to blood perfusion in specific tissues of a patient and to assess blood flow function in the patient. In some scenarios, the patient is not eligible for a CT perfusion imaging examination. For example, the patient's physical condition does not allow the injection of a high dose of contrast agent or the patient's condition is urgent, making it impossible to wait for perfusion imaging results. Additionally, medical facilities in the region where a patient is located may be relatively outdated, and the medical equipment required for CT perfusion imaging is lacking. These situations given as examples are often common, and thus the specialist is only able to judge the patient's vascular condition on the basis of a structural image obtained by CT vascular imaging and unable to judge the patient's vascular pathology accurately.

On this basis, the present disclosure provides a method for simulating blood perfusion, an apparatus for simulating blood perfusion, and an electronic device.

Embodiments of the present disclosure are described in detail below with reference to the drawings.

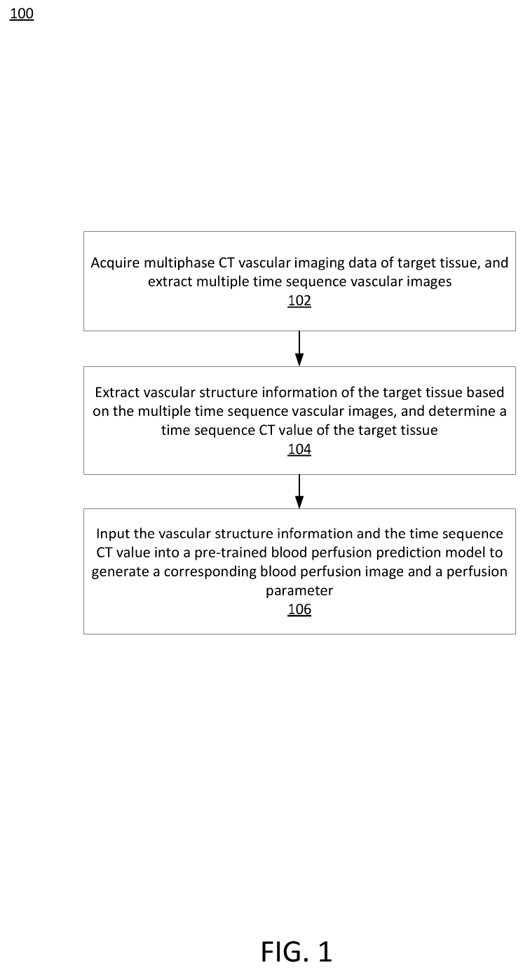

FIG. 1 shows a flowchart of a method 100 for simulating blood perfusion according to embodiments of the present disclosure.

Referring to FIG. 1, the method 100 for simulating blood perfusion comprises:
step 102, acquiring multiphase CT vascular imaging data of target tissue, and extracting multiple time sequence vascular images;
step 104, based on the multiple time sequence vascular images, extracting vascular structure information of the target tissue, and determining a time sequence CT value of the target tissue; and
step 106, inputting the vascular structure information and the time sequence CT value into a pre-trained blood perfusion prediction model, so as to generate a corresponding blood perfusion image and a perfusion parameter.

In the example of step 102, multiphase CT vascular imaging is also called multi-temporal-phase CT vascular imaging. Compared with ordinary single-phase CT vascular imaging techniques, this technique separately acquires CT vascular imaging data at multiple time points (time periods) rather than in a single time period. For this reason, multiphase CT vascular imaging has temporal resolution capability, i.e. is able to display the vascular engorgement state and hemodynamical variation more comprehensively so as to dynamically assess the collateral circulation state more effectively. In the case of diseases such as acute ischemic stroke for example, it is able to reflect the compensation level of collateral circulation more accurately.

In addition, in the method for simulating blood perfusion in embodiments of the present disclosure, the different time points at which CT vascular imaging data is acquired in the multiphase CT vascular imaging may include at least an arterial peak value phase, a venous peak value phase, and a venous late phase. The arterial peak value phase refers to a time point when the contrast agent reaches maximum density in the arterial system of the target tissue, at which time the imaging of the arterial system is most obvious. Thus, the imaging of the arterial system is clear, and the anatomical structure and blood flow state of the arteries can be clearly displayed. The venous peak value phase refers to a time point when the contrast agent reaches maximum density in the venous system of the target tissue, so the imaging of the venous system is most obvious. Thus, during this time period, the venous system has a good level of engorgement and the hemodynamical state and venous outflow condition of the veins can be better assessed. The venous late phase refers to a stable phase after the density of contrast agent in the venous system begins to fall and typically follows the venous peak value phase. In this time period, the imaging of the venous system becomes gradually weaker, but delayed imaging of the venous system can be observed so as to assess the state of venous collateral circulation. When the CT vascular imaging data simultaneously comprises data of the above three time points, the vascular state can be comprehensively assessed and dynamic variation of blood flow can be captured to a certain extent.

The question of whether the contrast agent reaches maximum density or remains stable in the arterial and venous systems is determined by variation in CT values in specific regions of the arterial and venous systems. The specific region may be e.g. a region of the venous/arterial system automatically identified in the target tissue or a region of interest (ROI) corresponding to the arterial/venous system of the target tissue designated by a specialist. Once the specific region has been determined, the variation of the CT value of the specific region with time can be acquired by scanning and recorded, to obtain a time-density curve for the specific region. When the CT value of the time-density curve reaches a peak value, the contrast agent can be judged to have reached maximum density. Additionally, when the CT value has fallen to a nearly unchanging level, the density of contrast agent can be judged to remain stable. An arterial density peak value threshold, a venous density peak value threshold and a venous density stability threshold may also be set in advance; when the acquired CT value reaches each of the abovementioned thresholds, the phase can be judged to be the arterial peak value phase, the venous peak value phase and the venous late phase, respectively. It is also possible, based on clinical data or experience, to judge that the arterial peak value phase, the venous peak value phase, and the venous late phase have been reached, and to acquire corresponding CT vascular imaging data, after delays of specific duration after the injection of contrast agent into the patient.

In the example of step 102, the target tissue is patient tissue to be examined. Since multiphase CT vascular imaging has the abovementioned temporal resolution capability, it has important value in decision-making for the diagnosis and treatment of diseases such as acute ischemic stroke and cerebrovascular disease. Therefore, the target tissue may typically be brain tissue of the patient. However, it will be understood that the method for simulating blood perfusion in embodiments of the present disclosure is not limited to the simulation of blood perfusion in brain tissue, and can also simulate blood perfusion information of tissues such as the heart and liver, so as to assist with the diagnosis and treatment of associated diseases.

On this basis, when multiphase CT vascular imaging data of target tissue is acquired, multiple time sequence vascular images can be extracted. Each acquisition time point (time period) comprises at least one corresponding vascular image.

In the example of step 104, vascular structure information of the target tissue is extracted from the multiple time sequence vascular images, and a time sequence CT value of the target tissue is determined.

In this example, based on the characteristics of CT vascular imaging technology, the time sequence CT value of the target tissue only comprises the CT value of the time point (time period) of acquisition. For other time points (e.g. time periods), acquisition may not be performed. The extracted vascular structure information may be a fused image generated by multiple time sequence vascular images or may be an image selected by screening from the multiple time sequence vascular images, which is best able to reflect the vascular structure of the target tissue. In addition, the vascular structure information may also be presented in other forms, for example, in the form of a group of CT values or a linear attenuation coefficient.

In the example of step 106, the vascular structure information and the time sequence CT value are inputted into a pre-trained blood perfusion prediction model so as to generate a corresponding blood perfusion image and a perfusion parameter.

In an example, the pre-trained blood perfusion prediction model used is not specifically defined. For example, a model based on a convolutional neural network (CNN), a long short-term memory network (LSTM), a gated recurrent unit (GRU) or a graph convolutional network, or a combination model, may be used.

In addition, in the example of step 106, the blood perfusion image generated may be a dynamic image or a static image generated on the basis of the vascular structure information of the target tissue extracted from the multiple time sequence vascular images and a perfusion parameter predicted by the blood perfusion prediction model. When the target tissue is the heart, a polar map capable of effectively reflecting myocardial perfusion may also be generated.

The acquired perfusion parameter may comprise a quantitative physiological blood perfusion measurement, for instance, blood volume (BV), i.e. the amount of blood contained in unit mass of tissue, in units of mL/100 g; blood flow (BF), meaning the amount of blood flowing through unit mass of tissue per unit time, in units of mL/min/100 g; mean transit time (MTT), meaning the average time taken for blood to pass through the capillary bed from the arterial side to the venous side, in units of seconds(s) or minutes (min). When the target tissue is brain tissue, the perfusion parameters acquired may be brain blood volume, brain blood flow, and mean transit time.

Compared with a conventional CT perfusion imaging method needing to acquire a time density curve of target tissue for an entire time period, the method in embodiments of the present disclosure only needs to acquire data at multiple predetermined time points (time periods), and can thus effectively reduce the dose of contrast agent required, reduce radiation exposure, and considerably shorten the time needed for data acquisition and post-processing, so as to improve imaging efficiency. In addition, the conventional CT perfusion imaging method requires special equipment which has a high cost; as a result, CT perfusion imaging has a high price, so has not achieved widespread application to a very high degree. The method in embodiments of the present disclosure relies on general-purpose CT vascular imaging equipment alone, so effectively reduces the cost of acquiring blood perfusion information.

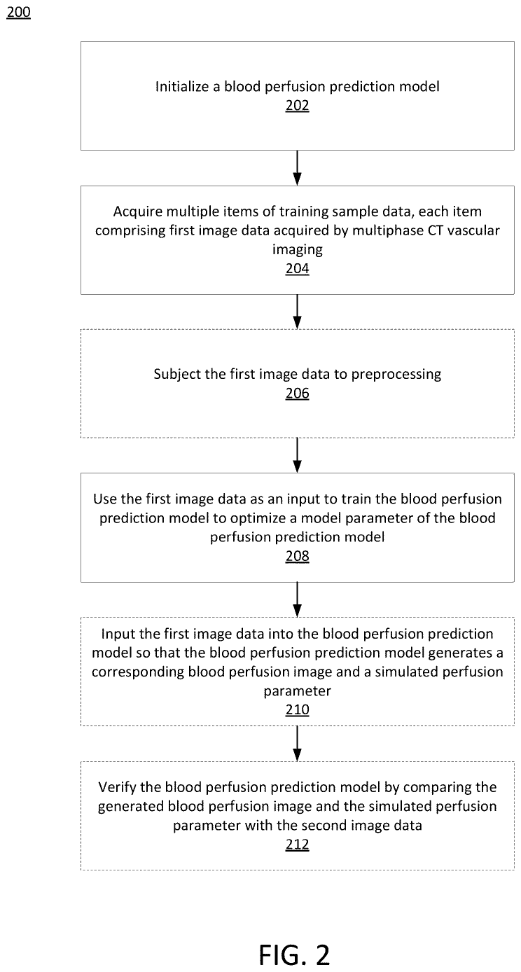

FIG. 2 shows a flowchart of a method 200 for training a blood perfusion prediction model. As shown in FIG. 2, according to some embodiments, the method 200 may comprise:
step 202, initializing a blood perfusion prediction model;
step 204, acquiring multiple items of training sample data, each item of training sample data comprising: first image data acquired by multiphase CT vascular imaging, the first image data may comprise vascular structure information and a time sequence CT value; and second image data acquired by corresponding CT perfusion imaging, the second image data may comprise a CT perfusion image and a perfusion parameter obtained by calculation on the basis of the CT perfusion image. The first image data and the second image data may be acquired at the same time and the same position; and
step 208, using the first image data as an input to train the blood perfusion prediction model, so as to optimize a model parameter of the blood perfusion prediction model.

In the example of step 202, initializing the blood perfusion prediction model may comprise initializing a model parameter, for example using a zero-initialization method to initialize all model parameters to zero, or using a method of random distribution such as uniform distribution or Gaussian distribution to randomly assign an initial value to a model parameter. The method of initialization is not specifically defined herein, and any suitable initialization strategy may be chosen according to the particular implementation.

In the example of step 204, each training sample comprises an image pair, e.g. an item of first image data acquired by multiphase CT vascular imaging, and an item of second image data acquired by corresponding CT perfusion imaging. To ensure one-to-one correspondence of data in the image pair, it is required that the first image data and the second image data in each image pair use the same acquisition position and the same acquisition time.

In the example of step 208, the first image data in the multiple training samples is used as an input to train the blood perfusion prediction model, so as to optimize the initialized model parameter.

As stated above, the multiphase CT vascular imaging has temporal resolution capability and is able to capture dynamic variation of blood flow, matching a dynamic characteristic of blood perfusion information. Thus, when the first image data acquired by multiphase CT vascular imaging is used as an input to train the blood perfusion prediction model, a blood perfusion prediction model with more accurate prediction ability can be obtained.

With continued reference to FIG. 2, the method 200 for training a blood perfusion prediction model may further comprise:
step 210, inputting the first image data into the blood perfusion prediction model, so that the blood perfusion prediction model generates a corresponding blood perfusion image and a simulated perfusion parameter;
step 212, verifying the blood perfusion prediction model by comparing the generated blood perfusion image and the simulated perfusion parameter with the second image data.

In the example of step 210 and step 212, the first image data as an input is not only able to optimize the model parameter, but may also be able to generate a corresponding blood perfusion image and a simulated perfusion parameter so that the blood perfusion prediction model can be verified by comparing the generated blood perfusion image and the simulated perfusion parameter with the second image data. This has the advantage that, since the second image data corresponds to the first image data, the second image data can assess the accuracy of model prediction, and further optimize the model parameter.

With continued reference to FIG. 2, before step 208, the method 200 for training a blood perfusion prediction model may further comprise:
step 206, subjecting the first image data to preprocessing, wherein the preprocessing comprises for instance image alignment, image noise reduction, and/or normalization.

In the example of step 206, subjecting the first image data to preprocessing can improve the prediction model training result and improve the accuracy with which the blood perfusion image and the simulated perfusion parameter are generated.

Image alignment may comprise aligning first image data acquired at different acquisition time points, so that corresponding points of multiple items of first image data precisely coincide in the same coordinate system; image alignment may also comprise aligning first image data with a pre-specified standard image. Image noise reduction is a process of reducing image noise by means of a specific algorithm or technique, with the objective of improving the quality of an image, so that it is clearer and easier to analyze. Normalization is a process of converting multiple items of first image data to a unified scale or range, with the objective of ensuring comparability and consistency of different images. For example, mean value normalization can be accomplished by subtracting the mean value from each pixel value of the first image data, then dividing by the standard deviation, so that the distribution of pixel values of the image is more balanced.

In addition, in some embodiments of the present disclosure, e.g. in the case of an already pre-trained blood perfusion prediction model, the model parameter may also be adjusted automatically on the basis of patient pathology data so that the individual physical condition of each patient can be taken into account comprehensively to more accurately predict blood perfusion information of the patient.

According to another aspect of the present disclosure, an apparatus for simulating blood perfusion is provided.

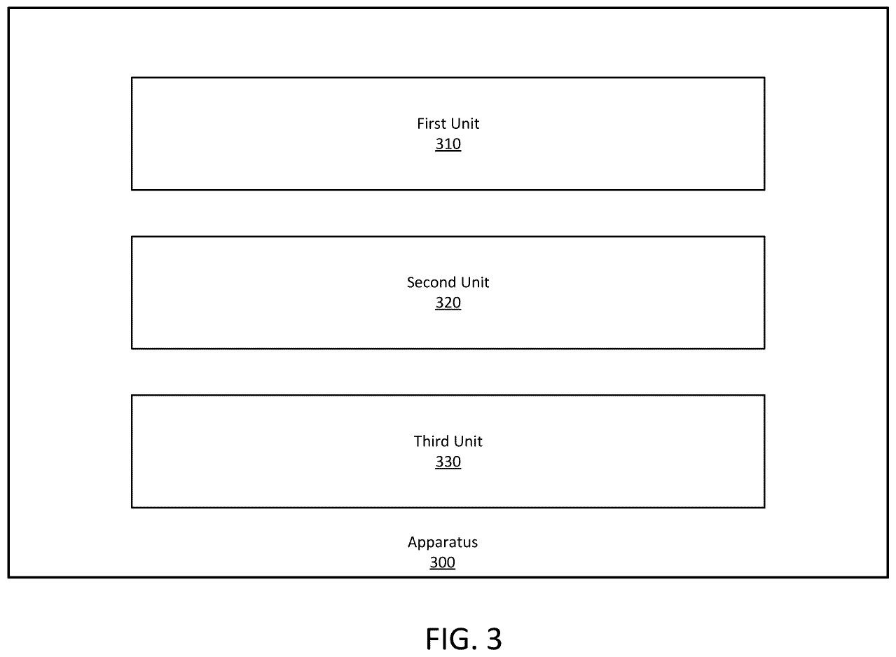

FIG. 3 shows a block diagram of the structure of an apparatus 300 for simulating blood perfusion according to embodiments of the present disclosure. As shown in FIG. 3, the apparatus 300 for simulating blood perfusion comprises:
a first unit 310 (also referred to herein as first processing circuitry), configured to acquire multiphase CT vascular imaging data of target tissue, and extract multiple time sequence vascular images;
a second unit 320 (also referred to herein as second processing circuitry), configured to extract vascular structure information of the target tissue on the basis of the multiple time sequence vascular images, and determine a time sequence CT value of the target tissue; and
a third unit 330 (also referred to herein as third processing circuitry), configured to input the vascular structure information and the time sequence CT value into a pre-trained blood perfusion prediction model, so as to generate a corresponding blood perfusion image and a perfusion parameter.

It will be understood that the units of the apparatus 300 shown in FIG. 3 may be configured to execute functions that correspond to steps 102-106 in the method 100 described with reference to FIG. 1. Thus, the operations, features, and advantages described above with respect to the method 100 are also applicable to the apparatus 300 and the units comprised therein. For the sake of conciseness, some operations, features and advantages are not described again here.

Although specific functions are discussed above with reference to specific units, it should be noted that the functions of each unit discussed herein may be divided into multiple units, and/or at least some of the functions of multiple units may be combined into a single unit. A specific unit discussed herein performing an action includes the specific unit itself performing the action or, alternatively, the specific unit invoking or otherwise accessing another component or unit that performs the action (or performs the action together with the specific unit). Therefore, a specific unit that performs an action may include the specific unit that performs the action itself and/or another unit that the specific unit invokes or otherwise accesses and that performs the action.

It should also be understood that various technologies may be described herein in the general context of a software/hardware element or a program unit. Each of the units described above with respect to FIG. 3 may be implemented in hardware or in hardware that combines software and/or firmware. For example, these units may be implemented as and/or configured to execute computer program code/instructions, and thus the units may be configured to be executed (when representing stored instructions) in one or more processors and stored in a computer-readable storage medium.

As another example, these units may be implemented as hardware logic/circuitry (e.g. one or more processors, processing circuitry, etc.). For example, in some embodiments, one or more of the first unit 310, the second unit 320 and the third unit 330 may be implemented together as hardware components in a System on Chip (SoC). The SoC may include an integrated circuit chip (which includes a processor (e.g., a central processing unit (CPU), a microcontroller, a microprocessor, a digital signal processor (DSP), etc.), a memory, one or more communication interfaces, and/or one or more components in other circuits), and may optionally execute the received program code and/or include embedded firmware to perform functions. Thus, the first unit 310, the second unit 320 and the third unit 330 may be implemented as any of such SoC components in this example.

According to another aspect of the present disclosure, an electronic device is provided, comprising: at least one processor; and a memory in communicative connection with the at least one processor; the memory stores instructions which can be executed by the at least one processor, and the instructions are executed by the at least one processor to enable the at least one processor to perform the method for simulating blood perfusion described above.

According to another aspect of the present disclosure, a non-transitory computer-readable storage medium storing a computer program is provided, wherein the computer program is configured to cause a computer to perform the method for simulating blood perfusion described above.

According to another aspect of the present disclosure, there is provided a computer program product comprising a computer program which, when executed by a processor, implements the method for simulating blood perfusion described above.

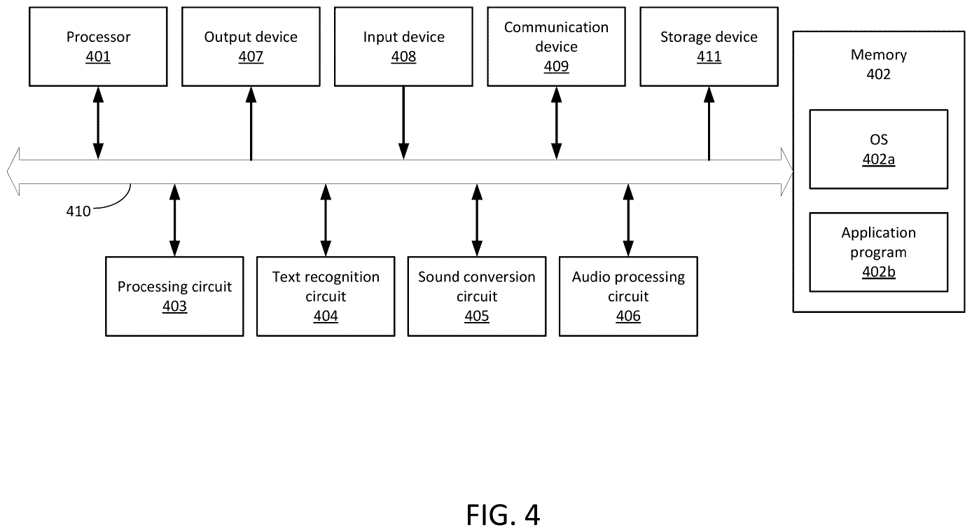

FIG. 4 is a block diagram showing an example of an electronic device 400 according to an exemplary embodiment of the present disclosure. It should be explained that the structure shown in FIG. 4 is merely an example. According to a specific manner of implementation, the electronic device of the present disclosure may only comprise one or more of the constituent parts shown in FIG. 4.

The electronic device 400 may be, for example, a general-purpose computer (e.g. a laptop computer, a tablet computer, or various other types of computer), a mobile phone, or a personal digital assistant. According to some embodiments, the electronic device 400 may be a cloud computing device, a smart device, etc. According to additional examples, the electronic device 400 may be implemented as an X-ray imaging device (or one or more portions thereof), such as a computed tomography (CT) device.

According to some embodiments, the electronic device 400 may be configured to process at least one of images, text and audio, and to transmit the result of this processing to an output device, so that it is provided to a user. For example, the output device may be a display screen or a device including a display screen, or may be a sound output device such as earphones, a speaker, or an oscillator. For example, the electronic device 400 may be configured to subject an image to target detection and transmit a result of target detection to the display device for display. The electronic device 400 may further be configured to subject the image to enhancement processing, and to transmit an enhancement result to the display device for display. The electronic device 400 may further be configured to recognize text in the image and to transmit a recognition result to the display device for display, and/or to convert the recognition result into sound data and transmit same to a sound output device for broadcast. The electronic device 400 may further be configured to recognize and process audio, to transmit a recognition result to the display device for display, and/or to convert a processing result into sound data and transmit same to the sound output device for broadcast.

The electronic device 400 may comprise an image processing circuit 403, and the image processing circuit 403 may be configured to subject the image to various types of image processing. The image processing circuit 403 may for example be configured to subject the image to at least one of the following types of image processing: image noise reduction, image normalization, image registration, image geometric correction, image feature extraction, detection and/or recognition of objects in the image, image enhancement, and detection and/or recognition of text contained in the image, etc.

The electronic device 400 may further include a text recognition circuit 404, and the text recognition circuit 404 is configured to perform text detection and/or recognition (e.g. OCR processing) of text regions in the image, so as to obtain text data. The text recognition circuit 404 may be implemented, for example, by a dedicated chip. The electronic device 400 may further include a sound conversion circuit 405, and the sound conversion circuit 405 is configured to convert the text data into sound data. The sound conversion circuit 405 may be implemented, for example, by a dedicated chip.

The electronic device 400 may further include an audio processing circuit 406, and the audio processing circuit 406 is configured to convert audio to text, so as to obtain text data corresponding to the audio. The audio processing circuit 406 may further be configured to process the text data corresponding to the audio. For example, the processing may include keyword extraction, intent recognition, intelligent recommendation, intelligent question and answer, etc. The audio processing circuit 406 may be implemented, for example, by a dedicated chip. The sound conversion circuit 405 may further be configured to convert an audio processing result into sound data, so as to be suitable for application scenarios such as voice assistants or virtual customer service.

One or more of the various circuits described above (e.g. the image processing circuit 403, the text recognition circuit 404, the sound conversion circuit 405 and the audio processing circuit 406) may be implemented using custom hardware, and/or using hardware, software, firmware, middleware, microcode, hardware description language or any combination thereof. For example, one or more of the various circuits described above may be implemented by using assembly language or hardware programming language (such as VERILOG, VHDL, C++) to program hardware (e.g. a programmable logic circuit including a field programmable gate array (FPGA) and/or a programmable logic array (PLA)), using the logic and algorithms according to the present disclosure.

According to some embodiments, the electronic device 400 may further comprise an output device 407. The output device 407 may be implemented for example as any suitable type of device used to present information, such as for example a display screen, a terminal with a display function, earphones, a loudspeaker, a vibrator and/or a printer, etc.

According to some embodiments, the electronic device 400 may further comprise an input device 408. The input device 408 may be implemented as any suitable type of device configured to input information to the electronic device 400, for example as various types of sensors, mouses, keyboards, touch screens, push buttons, control sticks, microphones and/or remote controllers, etc.

According to some embodiments, the electronic device 400 may further comprise a communication device 409. The communication device 409 may be implemented as any suitable type of device or system enabling communication with an external device and/or with a network, for example as a modem, a network card, an infrared communication device, a wireless communication device and/or a chipset, e.g. a Bluetooth device, a 802.11 device, a WiFi device, a WiMax device, a cellular communication device and/or analogs.

According to some embodiments, the electronic device 400 may further comprise a processor 401. The processor 401 may be implemented as any suitable type of processor and may include for example one or more general-purpose processors and/or one or more dedicated processors (e.g. special processing chips). The processor 401 may for example be, but is not limited to, a central processing unit (CPU), a graphics processing unit (GPU), or various types of dedicated artificial intelligence (AI) computing chips, etc.

The electronic device 400 may further comprise a working memory 402 and a storage device 411. The processor 401 may be configured to be able to acquire and execute computer-readable instructions stored in the working memory 402, the storage device 411 or another computer-readable medium, such as program code of an operating system 402a, program code of an application program 402b, etc. The working memory 402 and the storage device 411 are examples of computer-readable storage media used to store instructions, and the stored instructions can be executed by the processor 401 to implement the various functions described above. The working memory 402 may comprise both a volatile memory and a non-volatile memory (e.g. RAM, ROM, etc.). The storage device 411 may comprise a hard disk drive, a solid state drive, and a removable medium, including external and removable drives, memory cards, flash memories, floppy disks, optical disks (e.g. CD, DVD), storage arrays, network attached storage, storage area networks, etc. The working memory 402 and the storage device 411 may both be collectively referred to as memory or a computer-readable storage medium herein, and may be non-transitory media capable of storing computer-readable, processor-executable program instructions as computer program code, which may be executed by the processor 401 as a specific machine configured to implement the operations and functions described in the examples herein.

According to some embodiments, the processor 401 may control and schedule at least one of the image processing circuit 403, the text recognition circuit 404, the sound conversion circuit 405, the audio processing circuit 406 and various other apparatuses and circuits included in the electronic device 400. According to some embodiments, at least some of the constituent parts shown in FIG. 4 may be connected to and/or communicate with each other via a bus 410.

Software elements (programs) may be located in the working memory 402, including but not limited to an operating system 402a, one or more application programs 402b, a driver and/or other data and code.

According to some embodiments, instructions for performing the abovementioned control and scheduling may be included in the operating system 402a or one or more application programs 402b.

According to some embodiments, instructions for performing any of the method steps described in the present disclosure may be included in one or more application programs 402b, and the various modules of the electronic device 400 described above may be implemented by the processor 401 reading and executing the instructions of the one or more application programs 402b. In other words, the electronic device 400 may comprise the processor 401 and a memory storing a program (e.g. the working memory 402 and/or the storage device 411), the program comprising instructions which, when executed by the processor 401, cause the processor 401 to perform any of the methods as described in the various embodiments of the present disclosure.

According to some embodiments, some or all of the operations performed by at least one of the image processing circuit 403, the text recognition circuit 404, the sound conversion circuit 405, and the audio processing circuit 407 may be implemented by the processor 401 reading and executing instructions of one or more application programs 402b.

Executable code or source code of instructions of software elements (programs) may be stored in a non-transitory computer-readable storage medium (e.g. the storage device 411), and when executed, may be stored in the working memory 402 (possibly compiled and/or installed). Therefore, the present disclosure provides a computer-readable storage medium storing a program, the program comprising instructions which, when executed by a processor of an electronic device, cause the electronic device to perform any of the methods as described in the various embodiments of the present disclosure. According to another embodiment, executable code or source code of instructions of software elements (programs) may also be downloaded from a remote location.

It should also be understood that various modifications may be made according to specific requirements. For example, various circuits, units, modules or elements may also be implemented using custom hardware, and/or using hardware, software, firmware, middleware, microcode, hardware description language or any combination thereof. For example, some or all of the circuits, units, modules or elements included in the disclosed method and device may be implemented by using assembly language or hardware programming language (such as VERILOG, VHDL, C++) to program hardware (e.g. a programmable logic circuit including a field programmable gate array (FPGA) and/or a programmable logic array (PLA)), using the logic and algorithms according to the present disclosure.

According to some embodiments, the processor 401 in the electronic device 400 may be distributed on a network. For example, some processing may be performed using one processor, while other processing may be performed by another processor remote from that processor. Other modules of the electronic device 400 may also be similarly distributed. Thus, the electronic device 400 may be interpreted as being a distributed computing system that performs processing at multiple locations. The processor 401 of the electronic device 400 may also be a processor of a cloud computing system, or a processor in which a blockchain is incorporated.

Although embodiments or examples of the present disclosure have been described with reference to the drawings, it should be understood that the methods, systems and devices described above are merely exemplary embodiments or examples, and the scope of the present disclosure is not limited by these embodiments or examples, but is defined solely by the granted claims and the equivalent scope thereof. Various key elements in the embodiments or examples may be omitted or may be replaced by equivalent key elements thereof. In addition, the steps may be executed in an order different from that described in the present disclosure. Further, various key elements in the embodiments or examples may be combined in various ways. Importantly, as technology evolves, many key elements described here may be replaced by equivalent key elements appearing after the present disclosure.

In the present disclosure, unless otherwise stated, the use of the terms “first,” “second,” etc. to describe various key elements is not intended to define a positional relationship, time sequence relationship or importance relationship between these key elements; such terms are merely used to distinguish one element from another. In some examples, a first key element and a second key element may refer to the same instance of the element in question, whereas in some cases, based on the description in the context, they may also refer to different instances.

Terms used in the descriptions of the various examples in the present disclosure are merely intended to describe specific examples, and are not intended to impose restrictions. Unless clearly indicated otherwise in the context, if the quantity of a key element is not specifically defined, the key element may be one or more. In addition, the term “and/or” used in the present disclosure covers any one of the listed items and all possible combinations.

The various components described herein may be referred to as “units.” As noted above, such components may be implemented via any suitable combination of hardware and/or software components as applicable and/or known to achieve the intended respective functionality. This may include mechanical and/or electrical components, processors, processing circuitry, or other suitable hardware components configured to execute instructions or computer programs that are stored on a suitable computer readable medium. Regardless of the particular implementation, such units, as applicable and relevant, may alternatively be referred to herein as “circuitry,” “processors,” or “processing circuitry,” or otherwise as designated herein.

What is claimed is:

1. A method for simulating blood perfusion, comprising:
acquiring multiphase computerized tomography (CT) vascular imaging data of target tissue;
extracting, from the acquired multiphase CT vascular imaging data, multiple time sequence vascular images;
extracting, based on the multiple time sequence vascular images, vascular structure information of the target tissue;
determining, based on the multiple time sequence vascular images, a time sequence CT value of the target tissue; and
generating, based on inputting the vascular structure information and the time sequence CT value of the target tissue into a blood perfusion prediction model that simulates blood perfusion, a corresponding blood perfusion image and a blood perfusion parameter that represents a quantitative physiological blood perfusion measurement.

2. The method as claimed in claim 1, wherein the blood perfusion prediction model is trained by:
acquiring multiple items of training sample data, each item of training sample data comprising:
first image data acquired by the multiphase CT vascular imaging, the first image data comprising vascular structure information and a time sequence CT value; and
second image data acquired by corresponding CT perfusion imaging, the second image data comprising a CT perfusion image and a perfusion parameter obtained by calculation based on the CT perfusion image,
wherein the first image data and the second image data are acquired at a same time and a same position; and
training the blood perfusion prediction model using the first image data as an input so as to optimize a model parameter of the blood perfusion prediction model.

3. The method as claimed in claim 2, wherein training of the blood perfusion prediction model further comprises:
inputting the first image data into the blood perfusion prediction model so that the blood perfusion prediction model generates a corresponding blood perfusion image and a simulated perfusion parameter; and
verifying the blood perfusion prediction model by comparing the generated blood perfusion image and the simulated perfusion parameter with the second image data.

4. The method as claimed in claim 3, further comprising:
prior to inputting the first image data into the blood perfusion prediction model to perform training, subjecting the first image data to preprocessing,
wherein the preprocessing comprises image alignment, image noise reduction, and/or normalization.

5. The method as claimed in claim 1, wherein the multiphase CT vascular imaging data of the target tissue comprises CT vascular imaging data acquired at different time points, and
wherein the different time points comprise an arterial peak value phase, a venous peak value phase, and a venous late phase.

6. The method as claimed in claim 1, wherein the target tissue comprises brain tissue.

7. The method as claimed in claim 1, wherein the blood perfusion parameter generated by the blood perfusion prediction model comprises blood flow, blood volume, and mean transit time.

8. The method as claimed in claim 2, wherein the blood perfusion prediction model is configured to adjust the model parameter based on patient pathology data.

9. A non-transitory computer-readable medium having instructions stored thereon that, when executed by one or more processors of an electronic device, cause the electronic device to simulate blood perfusion by:
acquiring multiphase computerized tomography (CT) vascular imaging data of target tissue;
extracting, from the acquired multiphase CT vascular imaging data, multiple time sequence vascular images;
extracting, based on the multiple time sequence vascular images, vascular structure information of the target tissue;
determining, based on the multiple time sequence vascular images, a time sequence CT value of the target tissue; and
generating, based on inputting the vascular structure information and the time sequence CT value of the target tissue into a blood perfusion prediction model that simulates blood perfusion, a corresponding blood perfusion image and a blood perfusion parameter that represents a quantitative physiological blood perfusion measurement.

10. An electronic device, comprising:
a memory configured to store processor-readable instructions; and
a processor configured to execute the processor-readable instructions to simulate blood perfusion by:
acquiring multiphase computerized tomography (CT) vascular imaging data of target tissue;
extracting, from the acquired multiphase CT vascular imaging data, multiple time sequence vascular images;
extracting, based on the multiple time sequence vascular images, vascular structure information of the target tissue;
determining, based on the multiple time sequence vascular images, a time sequence CT value of the target tissue; and
generating, based on inputting the vascular structure information and the time sequence CT value of the target tissue into a blood perfusion prediction model that simulates blood perfusion, a corresponding blood perfusion image and a blood perfusion parameter that represents a quantitative physiological blood perfusion measurement.
